(19) United States
(12) Patent Application Publication
(10) Pub. No.: US 2020/0297325 A1
(43) Pub. Date: Sep. 24, 2020

(54) ULTRASONIC DIAGNOSTIC APPARATUS AND ULTRASONIC DIAGNOSTIC SYSTEM

(71) Applicant: KONICA MINOLTA, INC., Tokyo (JP)

(72) Inventors: Kenji KAZUI, Yokohama-shi (JP); Tomohiko SATSUKA, Tokyo (JP)

(73) Assignee: KONICA MINOLTA, INC., Tokyo (JP)

(21) Appl. No.: 16/794,625

(22) Filed: Feb. 19, 2020

(30) Foreign Application Priority Data
Mar. 19, 2019 (JP) 2019-050757

(51) Int. Cl. A61B 8/00; H02J 7/00

(52) U.S. Cl. CPC A61B 8/56; H02J 7/0048; H02J 7/0063; A61B 8/4427

(57) ABSTRACT

Provided is an ultrasonic diagnostic apparatus having a source power supplied from an external power supplier, and the ultrasonic diagnostic apparatus includes: a smoother that smoothens a source power which is supplied from the external power supplier having an external battery and on which a signal of battery information showing a state of the external battery is superimposed; a power supply that supplies the smoothened source power to each component within the ultrasonic diagnostic apparatus; a signal extractor that extracts the signal of the battery information from the source power supplied from the external power supplier; a converter that converts the extracted signal to the battery information; and a first hardware processor that performs control according to the converted battery information.

### CROSS-REFERENCE TO RELATED APPLICATIONS

The entire disclosure of Japanese Patent Application No. 2019-050757 filed on Mar. 19, 2019 is incorporated herein by reference in its entirety.

### BACKGROUND

### Technological Field

The present invention relates to an ultrasonic diagnostic apparatus and an ultrasonic diagnostic system.

### Description of the Related Art

Ultrasonography is capable of acquiring conditions of the heart and an embryo as ultrasonic images with a simple operation such as placing an ultrasonic probe on the body surface or from a body cavity with high safety, so that examinations can be repeatedly conducted. There is known an ultrasonic diagnostic apparatus used for conducting such ultrasonography.

As such an ultrasonic diagnostic apparatus, an ultrasonic diagnostic apparatus driven by batteries (cells) is known. Here, the configuration of a conventional ultrasonic diagnostic system 1C will be described by referring to FIG. 6. FIG. 6 is a block diagram showing the functional configuration of the conventional ultrasonic diagnostic system 1C.

As shown in FIG. 6, the ultrasonic diagnostic system 1C includes an ultrasonic diagnostic apparatus 10C and an external power supply unit 30C. The ultrasonic diagnostic apparatus 10C includes a controller 11C, a display 12, switches 14, 15, a power supply 16, a charging/discharging controller 17, and a battery 18. In the ultrasonic diagnostic apparatus 10C, each of other components such as an ultrasonic probe and an operator are not illustrated. The external power supply unit 30C includes an AC (Alternating Current) adapter 32 and a cable 37. The AC adapter 32 includes a plug 32a.

The ultrasonic diagnostic apparatus 10C is defined as a portable (hand-carried type) ultrasonic diagnostic apparatus that can be carried, for example. When the plug 32a is connected to an outlet of a commercial power supply, the AC adapter 32 converts an AC source power to a source power of a DC (Direct Current) voltage and supplies to the ultrasonic diagnostic apparatus 10C via the cable 37. At this time, when the battery 18 is not fully charged, the controller 11C turns on the switches 14 and 15 by the charging/discharging controller 17. The source power of the DC voltage is DC/DC converted by the power supply 16 and supplied to each component such as the controller 11C and the display 12 of the ultrasonic diagnostic apparatus 10C, and also supplied to the battery 18 to charge the battery 18.

When the plug 32a is not connected to the outlet of the commercial power supply, the controller 11C switches off the switch 14 and on the switch 15 by the charging/discharging controller 17. The source power of the DC voltage discharged from the battery 18 is DC/DC converted by the power supply 16 and supplied to each component of the ultrasonic diagnostic apparatus 10C such as the controller 11C and the display 12.

There are a lot of demands to be able to use the portable ultrasonic diagnostic apparatus for a long time only with the battery without connecting to the AC power supply. “Long time” means to be able to use for one day without connecting to the AC power supply, for example, which corresponds to about four operating hours when the power is frequently turned off and corresponds to about eight operating hours for unrestricted use without frequently turning off the power.

Therefore, there is known an ultrasonic diagnostic apparatus to which the source power is supplied from an external battery placed on a cart in addition to an AC-DC converter connected to a commercial power supply and a built-in battery (see JP 2011-78498A).

### SUMMARY

In order to operate for a long time, the size and mass of the external battery become great so that the external battery is provided as a separate unit that is isolated from the ultrasonic diagnostic apparatus. Considering the current energy density, the battery of the conventional ultrasonic diagnostic apparatus allows one-hour continuous operation or less, and the mass thereof is about 500 g. Therefore, in a case of eight-hour operation, the capacity of the battery becomes eight times the current capacity with a simple calculation so that the mass that is eight times the current mass also comes to be about 4 kg, which is too heavy to be mounted into the portable ultrasonic diagnostic apparatus.

Further, when employing a configuration in which an (external) battery as a separate unit is connected to the ultrasonic diagnostic apparatus, the ultrasonic diagnostic apparatus side handles the battery as an input of the source power of the DC voltage. In such case, the ultrasonic diagnostic apparatus receives supply of the source power of the DC voltage from the AC power supply (commercial power supply) via the AC adapter or supply of the source power of the DC voltage by connecting to the external battery for long-time use, so that it is not possible on the ultrasonic diagnostic apparatus side to know whether it is connected to the external battery or to the AC adapter.

As a typically used external battery, there is an uninterruptible power supply (UPS). The UPS is provided and utilized as a battery power supply between the AC power supply and the ultrasonic diagnostic apparatus, and designed to have a battery capacity capable of stably shutting down in case of a power failure. There are types of UPS with or without a function of giving a notification of the state of the AC power supply. In a case of having the function of giving such notification, it is common to use RS-232-C, USB (Universal Serial Bus), or LAN (Local Area Network) for a communication method of the notification. This is because an OS (Operating System) controls the whole actions in a case of a PC (Personal Computer) or the like and it is more advantageous to use the existing interface than utilizing a new interface considering the compatibility with the OS and because the UPS is not frequently detached. With the current UPS, USB or LAN is used in many cases.

Unlike the case of the UPS, the external battery for long-time use is designed to be utilized in an environment where the AC power supply is not available and to become free from troublesomeness of connecting to an AC power cable by utilizing the battery as the main power supply without connecting to the AC power supply. Therefore, there has been an increasing demand for a battery not only designed to connect the UPS as a connecting state and to enable stable shut down in a case of a power failure but also designed for long-time use.

In a case of using the external battery (outside battery), however, it is only possible on the ultrasonic diagnostic apparatus side to know a state where the DC power supply is supplied from outside but not possible to tell whether the connection with the ultrasonic diagnostic apparatus is the power supplied from the AC power supply or the power supplied from the external battery. Therefore, it may happen that the supplied source power is used for charging a built-in battery (inside battery) of the ultrasonic diagnostic apparatus, which may result in consuming the power wastefully.

Further, when the power is supplied from the external battery, the state of the remaining charge of the external battery is unknown. Therefore, the remaining charge of the external battery may suddenly run out, and known is the operable and useable time only with the remaining charge of the inside battery so that it may not be possible to achieve systematic use.

Further, even when connection signals are prepared by a connector of a communication method such as USB, LAN, or RS-232-C other than a power connector for connection between the ultrasonic diagnostic apparatus main body and the external battery as in the case of UPS, it is still necessary to connect a cable different from the power cable. Further, in a case of a portable external battery that can be carried, connection and removal of the cable occurs frequently so that there is a possibility of forgetting connection and there may also be troublesomeness felt when using.

It is an object of the present invention to notify information of the origin of the source power supplied to the ultrasonic diagnostic apparatus without providing a communication line separately from the power line.

To achieve at least one of the abovementioned objects, according to an aspect of the present invention, an ultrasonic diagnostic apparatus reflecting one aspect of the present invention is an ultrasonic diagnostic apparatus having a source power supplied from an external power supplier, and the ultrasonic diagnostic apparatus comprises:

a smoother that smoothens a source power which is supplied from the external power supplier having an external battery and on which a signal of battery information showing a state of the external battery is superimposed;

a power supply that supplies the smoothened source power to each component within the ultrasonic diagnostic apparatus;

a signal extractor that extracts the signal of the battery information from the source power supplied from the external power supplier;

a converter that converts the extracted signal to the battery information; and

a first hardware processor that performs control according to the converted battery information.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram showing a functional configuration of an ultrasonic diagnostic system according to an embodiment of the present invention;

FIG. 2A is a diagram showing the ultrasonic diagnostic system and a waveform of a source power when a signal is not superimposed on the source power;

FIG. 2B is a diagram showing the ultrasonic diagnostic system and a waveform of a source power when a signal is superimposed on the source power;

FIG. 3 is a chart showing a waveform of another source power;

FIG. 4 is a flowchart showing power supply control processing;

FIG. 5 is a diagram showing an ultrasonic diagnostic system according to a modification example and a waveform of a source power when a signal is superimposed on the source power; and

FIG. 6 is a block diagram showing a functional configuration of a conventional ultrasonic diagnostic system.

### DETAILED DESCRIPTION OF THE EMBODIMENTS

An embodiment and a modification example according to the present invention will be described in detail in order by referring to the accompanying drawings. However, the scope of the present invention is not limited to the disclosed embodiments.

### EMBODIMENT

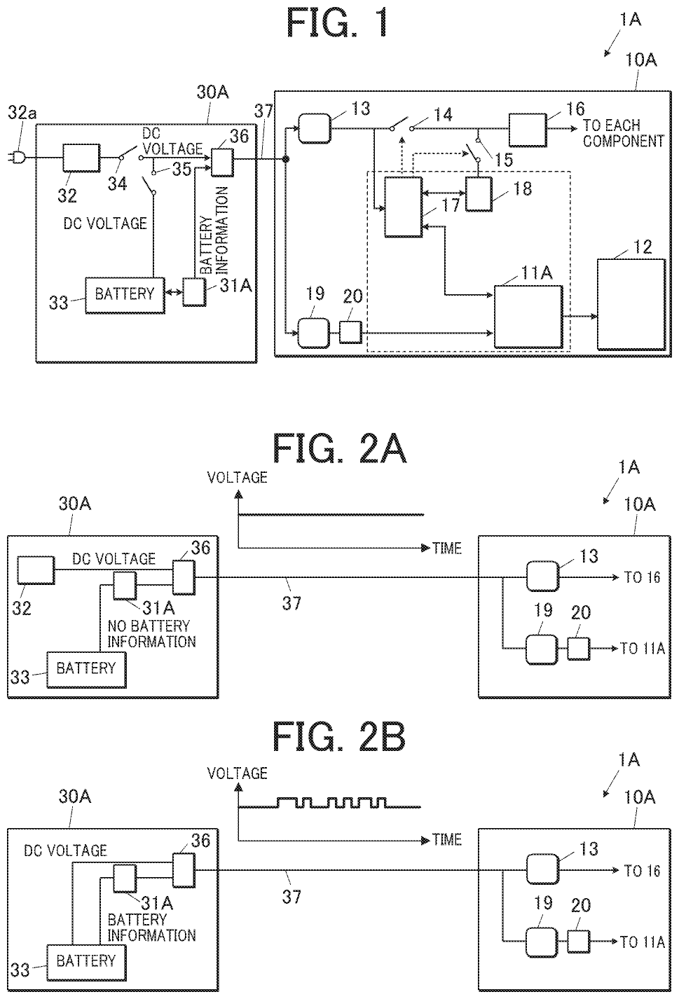

By referring to FIG. 1 to FIG. 4, the embodiment according to the present invention will be described. First, the configuration of an ultrasonic diagnostic system 1A according to the embodiment will be described by referring to FIG. 1. FIG. 1 is a block diagram showing the functional configuration of the ultrasonic diagnostic system 1A of the embodiment.

As shown in FIG. 1, the ultrasonic diagnostic system 1A includes: a portable ultrasonic diagnostic apparatus 10A that can be carried; and an external power supply unit 30A. The ultrasonic diagnostic apparatus 10A includes: a controller 11A as a first controller; a display 12 as an outputter; a smoothing circuit 13 as a smoother; switches 14, 15; a power supply 16; a charging/discharging controller 17; a battery 18; a signal pickup circuit 19 as a signal extractor; and a decoder 20 as a convertor. In the ultrasonic diagnostic apparatus 10A, illustrations of each of other components such as an ultrasonic probe that transmits/receives ultrasonic waves, a transmitter that generates drive signals for the ultrasonic probe, a receiver that receives reception signals from the ultrasonic probe, an image generator that generates ultrasonic image data from the reception signals, a display controller that displays the generated ultrasonic image data on the display 12, a storage that stores the ultrasonic image data and the like, an operator that receives operation input of users such as medical doctors, technologists, and the like are omitted.

The external power supply unit 30A includes: a controller 31A as a second controller; an AC adapter 32; a battery 33 as an external battery; switches 34, 35; a mixing circuit 36 as a superimposer; and a cable 37. The AC adapter 32 includes a plug 32a.

The controller 11A includes a CPU (Central Processing Unit), a RAM (Random Access memory), and a ROM (Read Only memory), reads out and expands various kinds of processing programs such as a system program stored in the ROM, and controls each component of the ultrasonic diagnostic apparatus 10A according to the expanded program. The ROM is configured with a nonvolatile memory such as a semiconductor, and stores a system program corresponding to the ultrasonic diagnostic apparatus 10A, various kinds of processing programs that can be executed on the system program, various kinds of data such as a gamma table, and the like. Those programs are stored in a form of program codes that can be read out by computers, and the CPU successively executes actions according to the program codes. The RAM is a volatile storage, and forms a work area for temporarily storing various kinds of programs executed by the CPU and the data related to those programs.

As the display 12, it is possible to employ display apparatuses such as an LCD (Liquid Crystal Display), a CRT (Cathode-Ray Tube) display, an organic EL (Electronic Luminescence) display, an inorganic EL display, a plasma display, and the like. The display 12 displays ultrasonic images and the like on a display screen according to image signals for display inputted from the display controller (not shown) under control of the controller 11A.

The smoothing circuit 13 is a circuit configured with a filter and the like for smoothing the voltage of the source power inputted from the external power supply unit 30A. The source power inputted from the external power supply unit 30A turns to a source power in which a signal component of battery information of the battery 33 is superimposed on a DC component. The smoothing circuit 13 removes the signal component by smoothing the voltage of the source power inputted from the external power supply unit 30A, and then outputs it to a latter stage as the source power with suppressed noise.

The switch 14 is a switching device provided between the smoothing circuit 13 and the power supply 16 for switching on/off of the current flown between the smoothing circuit 13 and the power supply 16. The switch 15 is a switching device provided between the battery 18 and the switch 14 as well as the power supply 16 for switching on/off of the current flown between the battery 18 and the smoothing circuit 13 or the power supply 16. Note that the switching device includes a switch configured with a semiconductor such as an FET (Field Effect Transistor).

The power supply 16 DC-DC converts the source power inputted from the smoothing circuit 13 of the battery 18 and distributes to each component of the ultrasonic diagnostic apparatus 10A. The power supply 16 is a power source that supplies the source power. The power supply 16 can be configured with a regulator (DC-DC converter, a series regulator). The regulator may be a circuit configured with a discrete component or may be configured with an IC (Integrated Circuit) or a circuit module.

The charging/discharging controller 17 is a circuit to which the source power is inputted from the smoothing circuit 13, and controls charging/discharging of the battery 18 by switching the switches 14, 15 according to the inputted source power and the battery information of the battery 18 under control of the controller 11A. The charging/discharging controller 17 can take various configurations according to the complication levels of the control, and can be achieved by a digital circuit, a small-scaled CPU such as a microcomputer, an IC dedicatedly used for controlling the power supply, or the like. The charging/discharging controller 17 is connected to the controller 11A and the battery 18 via an SM (System Management)-Bus.

The battery 18 is a built-in battery of the ultrasonic diagnostic apparatus 10A configured with a chargeable/dischargeable secondary battery such as a lithium ion battery, and has a function of outputting RSOC (Relative State Of Charge: relative remaining charge of the battery), fault-related information, and the like to the charging/discharging controller 17 as the battery information. RSOC is a ratio of the remaining capacity with respect to the full-charge capacity of the battery, and expressed as 0 to 100%. The fault-related information is information indicating an abnormal state such as information indicating that the battery 18 is too hot, information indicating that an excessive discharge current is flowing, and the like.

The signal pickup circuit 19 is a circuit that removes the DC component from the source power inputted from the external power supply unit 30A, regenerates DC, amplifies the signal component of the battery information of the battery 33 to be in a necessary amplitude, and then picks it up as a signal.

The decoder 20 decodes the signal inputted from the signal pickup circuit 19 in a decoding method corresponding to encoding of the controller 31A, and outputs it as the battery information of the battery 33 to the controller 11A.

The controller 31A is a controller that controls each component of the external power supply unit 30A, and has a function as an encoder that encodes the battery information outputted form the battery 33, a function of switching on/off the switches 34, 35, and the like. The controller 31A can be achieved by a CPU such as a microcomputer, an IC dedicatedly used for controlling the power supply, or the like.

The AC adapter 32 is an adapter that converts the alternating current commercial source power to the source power of the DC (Direct Current) voltage, and includes the plug 32a connected to an outlet of the commercial power supply. The battery 33 is an external battery of the ultrasonic diagnostic apparatus 10A configured with a chargeable/dischargeable secondary battery such as a lithium ion battery, and outputs RSOC, fault-related information, power information for grasping the output power of the battery 33, and the like to the controller 31A as the battery information. Further, it is defined that the capacity of the battery 33 is larger than the capacity of the battery 18, and is capable of outputting the source power for a relatively long time.

The switch 34 is a switching device provided between the AC adapter 32 and the mixing circuit 36 for switching on/off of the current flown between the AC adapter 32 and the mixing circuit 36. The switch 35 is a switching device provided between the battery 33 and the switch 34 as well as the mixing circuit 36 for switching on/off of the current flown between the battery 33 and the AC adapter 32 or the mixing circuit 36. Note that the switching device includes a switch configured with a semiconductor such as an FET.

The mixing circuit 36 is a circuit that adds and superimposes the voltage of the signal inputted from the controller 31A to the DC voltage of the source power inputted from the AC adapter 32 (switch 34) or the battery 33 (switch 35). Note that the mixing circuit 36 may also be configured as a circuit that subtracts the voltage of the inputted signal from the DC voltage of the inputted source power for superimposing.

There may be a case where about several A of current is flown in the power line that is inputted to the mixing circuit 36 from the AC adapter 32 or the battery 33. Therefore, the controller 31A decreases the pulse voltage (amplitude) of the signals generated by encoding the battery information of the battery 33 within a range with which malfunction does not occur and sets relatively larger intervals for the signals so as not to wastefully consume the power. Further, for the pulse voltage of the signals generated by encoding, it is also necessary suppress radiation and the like by controlling the frequency and the energy to be in a level with which no issues arise in terms of EMC (ElectroMagnetic Compatibility).

The cable 37 is a single power cable with one end being electrically connected to the mixing circuit 36 and the other end being electrically connected to an insertion port of the ultrasonic diagnostic apparatus 10A.

For each of the components of the ultrasonic diagnostic system 1A, a part of or the whole part of the functions of each of the functional blocks may be achieved as a hardware circuit such as an integrated circuit. The integrated circuit is an LSI (Large Scale Integration), for example, and the LSI may also be called an IC, a system LSI, a super LSI, or an ultra LSI depending on the integration levels. Further, the method of achieving the integrated circuit is not limited to LSI, and it is possible to employ a dedicated circuit or a multi-purpose processor or to use a reconfigurable processor that is capable of reconfiguring connection and setting of the circuit cells within the LSI. Further, a part of or the whole part of the functions of each of the functional blocks may be executed by software. In such case, the software is recorded in one or more recording medium such as a ROM, an optical disc, a hard disc, or the like, and the software is executed by a calculation processor.

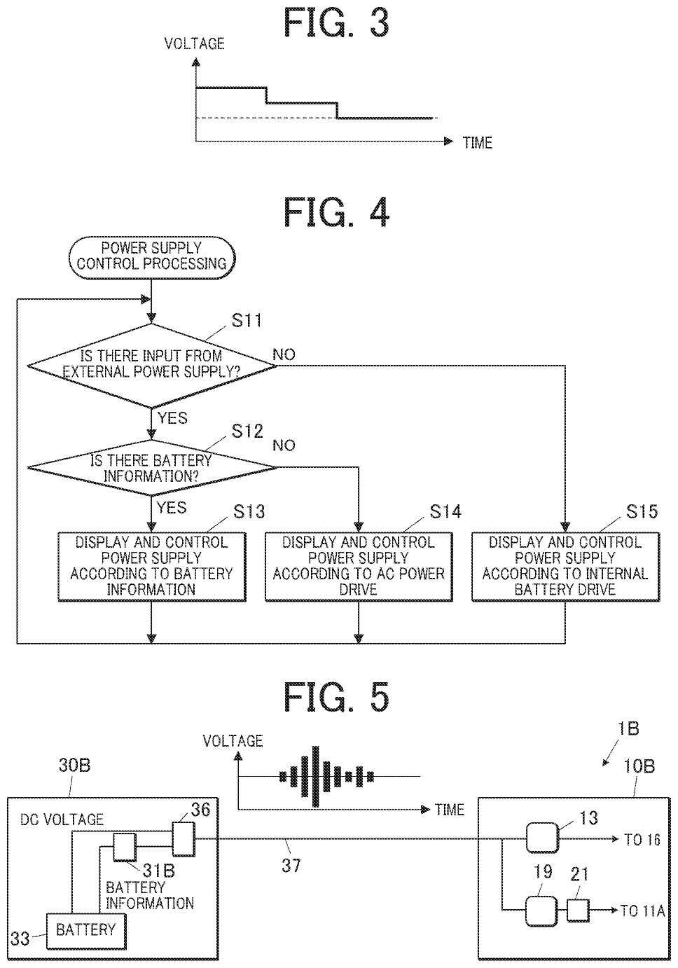

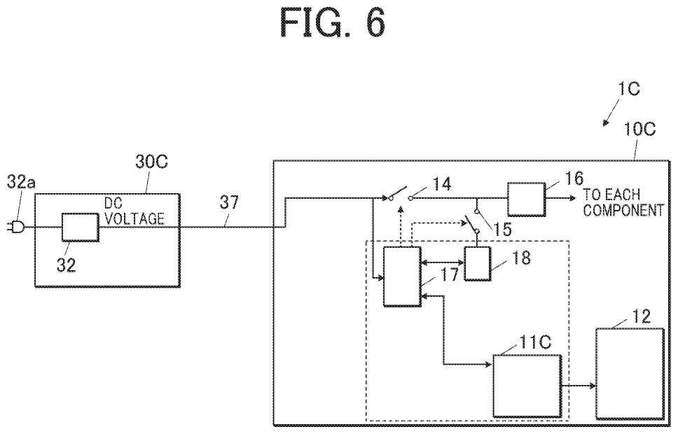

Next, actions of the ultrasonic diagnostic system 1A will be described by referring to FIG. 2A to FIG. 4. FIG. 2A is a diagram showing the ultrasonic diagnostic system 1A and a waveform of the source power when the signal is not superimposed on the source power. FIG. 2B is a diagram showing the ultrasonic diagnostic system 1A and a waveform of the source power when the signal is superimposed on the source power. FIG. 3 is a chart showing a waveform of another source power. FIG. 4 is a flowchart showing power supply control processing.

First, actions of the external power supply unit 30A side will be described by referring to FIG. 2A to FIG. 3. In the ultrasonic diagnostic system 1A, it is assumed that the external power supply unit 30A is connected in advance to the ultrasonic diagnostic apparatus 10A via the cable 37. For the power supply used for connection, it is assumed that the AC adapter 32 or the battery 33 as the external power supply is given priority over the battery 18 as the internal power supply of the ultrasonic diagnostic apparatus 10A and the adapter 32 is given priority over the battery 33.

As shown in FIG. 2A, described is a case where the plug 32a of the AC adapter 32 is connected to an outlet of a commercial power supply. The controller 31A switches on the switch 34 for supplying the power via the AC adapter 32. Further, the controller 31A refers to the battery information of the battery 33 and switches on the switch 35 when RSOC as the battery information is not 100% (not fully charged) and the fault-related information does not indicate an abnormal state. The AC adapter 32 outputs the source power of a constant DC voltage by the AC power supply. The source power of a constant DC voltage outputted from the AC power supply is inputted to the mixing circuit 36 and also supplied to the battery 33 to charge the battery 33. However, the battery 33 is charged without discharging, so that the controller 31A does not generate a signal corresponding to the battery information. The mixing circuit 36 outputs the source power of the DC voltage with a constant voltage value with respect to the time as in a waveform shown in FIG. 2A.

The source power of the constant DC voltage is inputted to the smoothing circuit 13 and the signal pickup circuit 19. The smoothing circuit 13 smoothens the inputted source power of the DC voltage and outputs to the power supply 16. The signal pickup circuit 19 does not extract the signal of the battery information from the inputted source power of the DC voltage since there is no battery information of the battery 33 and no signal of the battery information is outputted to the controller 11A via the decoder 20.

Further, in the ultrasonic diagnostic system 1A, the plug 32a of the AC adapter 32 is connected to the outlet of the commercial power supply, and the controller 31A switches on the switch 34 and off the switch 35 when the RSOC that is the battery information of the battery 33 is 100% (fully charged) or the fault-related information indicates an abnormal state. The source power of the constant DC voltage outputted from the AC adapter 32 is inputted to the mixing circuit 36. However, the battery 33 is unconnected to the mixing circuit 36, so that the controller 31A does not generate the signal corresponding to the battery information of the battery 33. Therefore, the mixing circuit 36 outputs the source power of the DC voltage with a constant voltage value with respect to the time as in the waveform shown in FIG. 2A.

As shown in FIG. 2B, described is a case where the plug 32a of the AC adapter 32 is not connected to the outlet of the commercial power supply and the battery 33 has a remaining charge (RSOC>0%). The controller 31A switches off the switch 34 for preventing backflow from the battery 33 to the AC adapter 32. Further, the controller 31A switches on the switch 35. The source power of the constant DC voltage discharged from the battery 33 is inputted to the mixing circuit 36. When the output power value of the battery 33 reaches a prescribed value or more according to the power information of the battery information of the battery 33, the controller 31A generates a signal by encoding the battery information outputted from the battery 33 and outputs the signal to the mixing circuit 36.

At this time, the mixing circuit 36 outputs the source power in which a signal of a constant amplitude corresponding to the battery information of the battery 33 is superimposed (added) on the DC voltage with a constant voltage value with respect to the time as in a waveform shown in FIG. 2B, for example.

The power consumption of the ultrasonic diagnostic apparatus 10A also becomes high when the voltage signal is superimposed on the source power inputted to the ultrasonic diagnostic apparatus 10A. Therefore, when the output power value of the battery reaches the prescribed value or more, the signal is superimposed on the source power and the frequency of communication and the signal amount are decreased to also decrease the power consumption of the ultrasonic diagnostic apparatus 10A, and to shorten the time until the ultrasonic diagnostic apparatus 10A side recognizes by the superimposed signal that the battery 33 is connected (the source power is discharge) to the ultrasonic diagnostic apparatus 10A.

Further, the battery information can be transmitted by determining a transmission/reception protocol according to the signal defined in advance and encoding the battery information according to the transmission/reception protocol by the controller 31A to generate the signal. The controller 31A encodes the battery information of the battery 33 and controls the frequency, the amplitude, the pulse width, and the like to generate the signal. For example, the number of pulses is determined according to the signal amount transmitted at once. For example, it may be defined that the RSOC is 10% or less with the source power from the battery 33 with a single pulse and that the RSOC is 10% or more and 50% or less with the source power from the battery 33 with two pulses. When the controller 31 determines in advance to transmit the battery information of the battery 33 at prescribed intervals such as once in every 10 minutes, the ultrasonic diagnostic apparatus 10A side can correspond to that.

The source power on which the signal of the battery information is superimposed is inputted to the smoothing circuit 13 and the signal pickup circuit 19. The smoothing circuit 13 smoothens the inputted source power of the DC voltage and outputs to the power supply 16. The signal pickup circuit 19 picks up the signal of the battery information from the inputted source power of the DC voltage. The decoder 20 decodes the signal extracted by the signal pickup circuit 19 and outputs to the controller 11A.

Further, the controller 31A may be configured to generate signals with DC voltage levels of a plurality of stages as UNREG voltages (Unregulated: power supply of inconstant voltages) as shown in FIG. 3 by encoding.

Further, the controller 31A may also be configured to generate signals coded by other communication methods such as CAN (Controller Area Network), 8b/10b, and the like. A CAN signal is a signal of a bus standard of the communication employed for communication and the like inside automobiles. Note that 8b/10b is a method which converts 8-bit data to 10-bit data and transfers the converted data, and a clock signal is embedded in a signal line to transfer the data and the clock via a same wiring. This method is also employed for Ethernet (R) (LAN), PCIe (Peripheral Component Interconnect express), SATA (Serial Advanced Technology Attachment), and USB 3.0 (SS (Super Speed)). While physical layers and transfer rates are different from respective standardized interfaces, it is possible to use the existing communication protocols as they are by employing those code methods as the signal transmission method, and those coding methods can be used when a large-volume of communication is required.

Next, voltage control processing executed by the controller 31A will be described as actions on the ultrasonic diagnostic apparatus 10A side by referring to FIG. 4. First, the controller 11A determines whether or not there is input of the source power from the external power supply unit 30A according to presence of the source power inputted to the charging/discharging controller 17 (step S11). If there is input of the external source power (YES in step S11), the controller 11A determines whether or not the battery information of the battery 33 is included in the source power according to the presence of the decoded signal of the battery information from the decoder 20 (step S12).

If the battery information is included (YES in step S12), the controller 11A determines that the source power is external battery drive supplied from the battery 33, generates and displays on the display 12 the display information indicating that the source power is the external battery drive and showing the inputted battery information of the battery 33, performs power supply control by on/off controlling the switches 14, 15 according to the inputted battery information of the battery 18 by the charging/discharging controller 17 (step S13), and shifts to step S11.

The display information of step S13 includes information showing the RSOC such as characters and figures and warning information corresponding to the fault-related information within the battery information of the battery 33, for example. Further, in step S13, the controller 11A controls to switch on the switch 14 and off the switch 15 by the charging/discharging controller 17, determining that charging the battery 18 from the battery 33 is wasteful.

If the RSOC in the battery information of the battery 18 is not 100% (not fully charged) and the fault-related information does not indicate an abnormal state in step S13, the controller 11A may be configured to switch on the switches 14, 15 by the charging/discharging controller 17 to supply the source power to each component of the ultrasonic diagnostic apparatus 10A via the smoothing circuit 13 and the power supply 16 and to charge the battery 18. With such configuration, if the RSOC in the battery information of the battery 18 is 100% (fully charged) or the fault-related information indicates an abnormal state in step S13, the controller 11A switches on the switch 14 and off the switch 15 by the charging/discharging controller 17 to supply the source power to each component via the smoothing circuit 13 and the power supply 16.

If the battery information is not included (NO in step S12), the controller 11A determines that the source power is AC drive supplied from the AC adapter 32, generates and displays on the display 12 the display information indicating that the source power is the AC drive and showing the battery information of the battery 18, performs power supply control by on/off controlling the switches 14, 15 according to the inputted battery information of the battery 18 by the charging/discharging controller 17 (step S14), and shifts to step S11.

If the RSOC in the battery information of the battery 18 is not 100% (not fully charged) and the fault-related information does not indicate an abnormal state in step S13, the controller 11A switches on the switches 14, 15 by the charging/discharging controller 17 to supply the source power to each component of the ultrasonic diagnostic apparatus 10A via the smoothing circuit 13 and the power supply 16 and to charge the battery 18. Further, if the RSOC in the battery information of the battery 18 is 100% (fully charged) or the fault-related information indicates an abnormal state in step S13, the controller 11A switches on the switch 14 and off the switch 15 by the charging/discharging controller 17 to supply the source power to each component via the smoothing circuit 13 and the power supply 16.

If there is no input of the external source power (NO in step S11), the controller 11A determines that the source power is internal battery drive supplied from the battery 18, generates and displays on the display 12 the display information indicating that the source power is the internal battery drive and showing the inputted battery information of the battery 18, performs power supply control by on/off controlling the switches 14, 15 according to the inputted battery information of the battery 18 by the charging/discharging controller 17 (step S15), and shifts to step S11.

The display information of step S15 includes information showing the RSOC such as characters and figures and warning information corresponding to the fault-related information within the battery information of the battery 33, for example. Further, if the fault-related information of the battery information of the battery 18 does not indicate an abnormal state in step S15, the controller 11A switches off the switch 14 and on the switch 15 by the charging/discharging controller 17 to supply the source power to each component. If the fault-related information of the battery information of the battery 18 indicates an abnormal state in step S15, the controller 11A switches off the switch 14 as well as the switch 15 by the charging/discharging controller 17 to stop supply of the source power to each component, for example.

When the RSOC of the battery becomes small (when becomes lower than a prescribed value, for example), it is also possible for the controller 11A to take an action for reducing the power consumption. For example, as necessary, the controller 11A tries to reduce the power consumption by shifting to “standby” as a standby power mode or by shifting to “resume” or the like with which the information of the current state is temporarily saved in the storage as a low power consumption mode according to the RSOC.

According to the embodiment as described above, the ultrasonic diagnostic apparatus 10A has the source power supplied from the external power supply unit 30A. The ultrasonic diagnostic apparatus 10A includes: the smoothing circuit 13 that smoothen the source power which is supplied from the external power supply unit 30A having the external battery 33 and on which the signal of the battery information showing the state of the battery 33 is superimposed; the power supply 16 that supplies the smoothened source power to each component inside the ultrasonic diagnostic apparatus 10A; the signal pickup circuit 19 that extracts the signal of the battery information of the battery 33 from the source power supplied from the external power supply unit 30A; the decoder 20 that decodes the extracted signal into the battery information; and the controller 11A that performs control according to the decoded battery information.

Therefore, it is possible to notify the battery information of the battery 33 as the origin of the source power supplied to the ultrasonic diagnostic apparatus 10A without providing a communication line separately from the cable 37 as the power line, and the ultrasonic diagnostic apparatus 10A side can recognize the battery information of the battery 33.

Further, the controller 11A generates and displays the display information according to the decoded battery information on the display 12. Therefore, because the battery information of the battery 33 is notified, the user can recognize the battery information, the useable time and the like based thereupon, and can take a necessary measure.

Further, the battery information includes the remaining charge information of the battery 33. The controller 11A shifts to the low power consumption mode such as “standby” or “resume” according to the RSOC in the battery information of the decoded signal. This makes it possible to save the power of the ultrasonic diagnostic apparatus 10A according to the RSOC.

Further, the controller 11A determines whether or not there is extracted (decoded signal) when the source power is supplied from the external power supply unit 30A, determines that the source power is supplied from the battery 33 when there is the extracted signal, and determines that the source power is supplied from the AC power of the AC adapter 32 of the external power supply unit 30A when there is no extracted signal. Therefore, it is possible to recognize that there is no connection with the battery 33 based on the fact that there is no superimposed signal on the source power, and the controller 11A can perform display control of the display information and the power control according to the supply (the AC adapter 32 or the battery 33) of the source power.

Further, the ultrasonic diagnostic system 1A includes the ultrasonic diagnostic apparatus 10A and the external power supply unit 30A. The external power supply unit 30A includes: the controller 31A that generates the signal from the battery information inputted from the battery 33; and the mixing circuit 36 that superimposes the generated signal on the source power discharged from the battery 33. Therefore, it is possible to notify the battery information of the battery 33 as the origin of the source power supplied to the ultrasonic diagnostic apparatus 10A without providing a communication line separately from the cable 37 as the power line.

Further, the mixing circuit 36 adds or subtracts the generated signal to superimpose on the source power that is discharged from the battery 33. Therefore, the signal can be easily superimposed on the source power.

Further, the controller 31A generates the signal by controlling the frequency, the amplitude, and the pulse width. Therefore, the signal can be generated appropriately.

Further, the controller 31A generates the signal by coding the battery information. Therefore, the signal with a large amount of information can be easily generated.

Further, the controller 31A determines whether or not the output power of the battery 33 has reached a prescribed threshold value or more, and generates a signal from the battery information inputted from the battery 33 when determined to have reached the prescribed threshold value or more. Therefore, by not constantly sending the information between the battery 33 and the ultrasonic diagnostic apparatus 10A but by determining discharge of the source power from the battery 33 based on the power consumption and starting communication thereupon, it is possible to decrease the power consumption of the ultrasonic diagnostic apparatus 10A by decreasing waste of the power caused by constantly sending the information and also possible to lower the risk of having noise or the like generated by the information communication. Further, it is also possible to shorten the time until the ultrasonic diagnostic apparatus 10A side recognizes with the superimposed signal that the battery 33 is connected (the source power is discharged) to the ultrasonic diagnostic apparatus 10A.

### Modification Example

A modification example of the embodiment will be described by referring to FIG. 5. FIG. 5 is a diagram showing an ultrasonic diagnostic system 1B and a waveform of a source power when a signal is superimposed on the source power.

As shown in FIG. 5, in the modification example, the ultrasonic diagnostic system 1B is used instead of the ultrasonic diagnostic system 1A of the embodiment. The ultrasonic diagnostic system 1B includes an ultrasonic diagnostic apparatus 10B and an external power supply unit 30B. The ultrasonic diagnostic apparatus 10B has a configuration in which the controller 31A of the ultrasonic diagnostic apparatus 10A according to the embodiment is replaced with the controller 31B. The external power supply unit 30B has a configuration in which the decoder 20 according to the embodiment is replaced with a demodulator 21 as a converter.

The controller 31B is a controller that controls each component of the external power supply unit 30B, and has a function as a modulator that modulates the battery information outputted from the battery 33, a function of switching on/off the switches 34, 35, and the like. The controller 31B can be achieved by a CPU such as a microcomputer, an IC dedicatedly used for controlling the power, or the like. As the modulation method of the battery information of the battery 33, it is possible to employ FM (Frequency Modulation), AM (Amplitude Modulation), or the like as the modulation method using analog signals, for example. Thereby, the battery information is transmitted as analog signals, so that it is possible to limit the frequency to be mixed and to suppress the influence of the noise imposed upon the circuit of the ultrasonic diagnostic system 1B.

The demodulator 21 demodulates the signal inputted from the signal pickup circuit 19 with a demodulation method corresponding to the modulation method of the controller 31B, and outputs to the controller 11A as the battery information of the battery 33.

According to the modification example as described above, the controller 31B modulates the battery information of the battery 33 to generate an analog signal. The demodulator 21 demodulates the analog signal extracted from the source power to convert into the battery information. Therefore, the signal can be appropriately generated. At the same time, the battery information is transmitted as the analog signal, so that it is possible to limit the frequency to be mixed and to suppress the influence of the noise imposed upon the circuit of the ultrasonic diagnostic system 1B.

Note that the embodiment and the modification example described above are examples of the preferred ultrasonic diagnostic apparatus and ultrasonic diagnostic system according to the present invention, and the present invention is not limited to those.

For example, while the embodiment and the modification example are configured such that the controller 11A generates and displays on the display 12 the display information based on the battery information of the battery 33, the configuration is not limited to that. It is also possible to employ a configuration in which the ultrasonic diagnostic apparatus 10A or 10B includes a sound outputter such as an amplifier or a speaker as an outputter, and the controller 11A generates sound information based on the battery information of the battery 33 and has the sound outputter output the sound.

Further, detailed configurations and detailed actions of each component configuring the ultrasonic diagnostic systems 1A and 1B according to the embodiment and the modification example described above can be changed as necessary without departing from the range of the gist of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus having a source power supplied from an external power supplier, the ultrasonic diagnostic apparatus comprising:
a smoother that smoothens a source power which is supplied from the external power supplier having an external battery and on which a signal of battery information showing a state of the external battery is superimposed;
a power supply that supplies the smoothened source power to each component within the ultrasonic diagnostic apparatus;
a signal extractor that extracts the signal of the battery information from the source power supplied from the external power supplier;
a converter that converts the extracted signal to the battery information; and
a first hardware processor that performs control according to the converted battery information.

2. The ultrasonic diagnostic apparatus according to claim 1, wherein the first hardware processor generates output information based on the converted battery information and has an outputter output the output information.

3. The ultrasonic diagnostic apparatus according to claim 1, wherein:
the battery information includes remaining charge information of the external battery; and
the first hardware processor shifts to a low power consumption mode according to the remaining charge information in the battery information of the converted signal.

4. The ultrasonic diagnostic apparatus according to claim 1, wherein the first hardware processor determines whether or not there is the extracted signal when the source power is supplied from the external power supplier, determines that the source power is supplied from the external battery when there is the extracted signal, and determines that the source power is supplied from an alternating current power supply of the external power supplier when there is no extracted signal.

5. An ultrasonic diagnostic system, comprising
the ultrasonic diagnostic apparatus according to claim 1, and
the external power supplier, wherein:
the external power supplier includes:
a second hardware processor that generates the signal from the battery information inputted from the external battery; and
a superimposer that superimposes the generated signal on the source power discharged from the external battery.

6. The ultrasonic diagnostic system according to claim 5, wherein the superimposer adds or subtracts to superimpose the generated signal on the source power discharged from the external battery.

7. The ultrasonic diagnostic system according to claim 5, wherein the second hardware processor generates the signal by controlling a frequency, an amplitude, and a pulse width.

8. The ultrasonic diagnostic system according to claim 5, wherein the second hardware processor generates the signal by coding the battery information.

9. The ultrasonic diagnostic system according to claim 5, wherein:
the second hardware processor generates the signal in an analog form by modulating the battery information; and
the converter demodulates the extracted analog signal to convert to the battery information.

10. The ultrasonic diagnostic system according to claim 5, wherein the second hardware processor determines whether or not output power of the external battery has reached a prescribed threshold value or more, and generates the signal from the battery information inputted from the external battery when determined to have reached the prescribed threshold value or more.
